(19) United States
(12) Patent Application Publication
(10) Pub. No.: US 2007/0212790 A1
(43) Pub. Date: Sep. 13, 2007

(54) Method for monitoring feeds to catalytic cracking units by near-infrared spectroscopy

(75) Inventors: William T. Welch, Ashland, KY (US); James F. Hoffman, Huntington, WV (US); Brian K. Wilt, Flatwoods, KY (US); Roy Roger Bledsoe, Huntington, WV (US); Michael B. Sumner, Kenova, WV (US); Jeff Sexton, Findlay, OH (US)

Correspondence Address: EMCH, SCHAFFER, SCHAUB & PORCELLO CO, P O BOX 916, ONE SEAGATE SUITE 1980, TOLEDO, OH, 43697, US

(73) Assignee: Marathon Petroleum Company LLC

(21) Appl. No.: 11/656,320

(22) Filed: Jan. 19, 2007

Related U.S. Application Data
(60) Provisional application No. 60/781,840, filed on Mar. 13, 2006.

(51) Int. Cl. G01N 33/00

(52) U.S. Cl. 436/139

(57) ABSTRACT

A monitoring of catalytic cracking processing is provided which uses near infrared (NIR) analysis to characterize cracking feeds, intermediates and products for chemical and physical properties such as saturates, monoaromatics, diaromatics, triaromatics, tetraaromatics, polar aromatics, total aromatics, thiophenes, distillation points, basic nitrogen, total nitrogen, API gravity, total sulfur, MCRT and % coker gasoil and the resulting characterization thereof. The NIR results can be used in FCC simulation software to predict unit yields and qualities.

### CROSS REFERENCE TO RELATED APPLICATION

This application is a conversion of and claims the benefit of U.S. provisional patent application Ser. No. 60/781,840 filed Mar. 13, 2006.

### TECHNICAL FIELD

This invention relates to monitoring a catalyzed cracking unit (FCC) and feedstock selection by near infrared spectroscopy. More specifically, the present invention relates to the monitoring of catalytic cracking processes for producing lower molecular weight products from hydrocarbon feeds and the monitoring of such processes by NIR spectroscopy.

### BACKGROUND OF THE INVENTION

Near IR spectroscopy has been used in the past to determine chemical and physical properties of petroleum hydrocarbon mixtures. This includes using the NIR results with refinery processes including gasoline blenders and catalytic reforming units. It is a quick, non-destructive analytical technique that is correlated to primary test methods using a multivariate regression analysis algorithm such as partial least squares or multiple linear regression. It has been used in laboratory and on-line settings to predict properties of refinery blender streams, finished gasoline and diesel fuel.

Optimization and design of catalytic cracking process units all benefit from kinetic models which describe the conversion of feeds to products. In order to properly describe the effects of changes in feed composition, such models require descriptions of the feed in terms of constituents which undergo similar chemical reactions in the cracking unit. For design and optimization studies, a protocol which involves off-line feed analysis taking weeks or even months to provide a feed description. However, for monitoring a process unit, a rapid and convenient method for characterization of the feeds to catalytic units is required. Current analytical techniques to characterize FCC feeds are time consuming and labor intensive. The invention provides the needed FCC feed characterization using only a fraction of the time and labor involved in performing the standard analyses.

Other objects and advantages of the present invention will become apparent to those skilled in the art upon a review of the following detailed description of the preferred embodiments and the accompanying drawings.

### SUMMARY OF THE INVENTION

A near IR (NIR) spectrophotometer can be used to collect spectra of fluid catalytic cracking (FCC) feed stocks. The collected NIR spectral data was correlated to traditional laboratory tests including HPLC Heavy Distillate Analyzer (HDA) results of aromatic core type (1-ring core, 2-ring core, 3-ring core, 4-ring core and polars), ASTM D2887 high temperature simulated distillation, basic nitrogen, total nitrogen, API gravity, total sulfur, MCRT, and percent of Coker gas oil in Vacuum Gas Oil (VGO). The NIR can be used to monitor the FCC feed stocks quality more quickly and efficiently than performing the lab tests.

Furthermore, certain critical wavelengths have been found to be of special value in determining the optimum operation of a catalytic cracking unit (FCCU).

The present invention provides a process for analyzing catalytic cracking hydrocarbon feeds, intermediates and products exhibiting absorption in the near infrared (NIR) region. The process steps include:
a) measuring absorbances of the feed using a spectrometer measuring absorbances at wavelengths within the range of about 780-4000 nm, e.g., 780-2500 nm, and outputting an emitted signal indicative of said absorbance;
b) subjecting the NIR spectrometer signal to a mathematical treatment (e.g., derivative, smooth, baseline correction) of the emitted signal.
c) processing the emitted signal or the mathematical treatment using a defined model to determine the chemical or physical properties of feeds, intermediates or products and outputting a processed signal; and
d) monitoring in response to the processed signal, at least one parameter of the catalytic cracking feed, intermediate or product.

### BRIEF DESCRIPTION OF THE DRAWINGS

FIG. 1 is a schematic diagram of an FCC unit comprising a reactor and a regenerator showing the control system of the present invention in place for operating that FCC unit.

FIG. 2 is a Table which shows samples, including hydrotreater charges and products and FCC feeds used to control on-line weight percents of each hydrocarbon class.

FIG. 3 is a plot showing FCC feed sulfur under different operating philosophies.

FIG. 4 is a graph of a catalyst cycle life curve.

FIG. 5 is a graph of an FCC feed upset showing high SO_{X}.

FIG. 6 is a table of a neural network for on-line control of SO_{X} emissions.

FIG. 7 is a graph of the use of NIR on FCC hydrotreating.

FIG. 8 is a graph showing NIR predicted results versus Lab results for % Coker Gas Oil.

FIGS. 9 and 10 are graphs showing typical monitoring plots for feedstock quality and product yields.

### DETAILED DESCRIPTION OF THE INVENTION

The preferred embodiment of monitoring on-line in response to the processed signal is carried out with the following steps.

The preferred process includes the step of using NIR measuring to provide real time optimization of (RTO) FCC monitoring.

The process also may include the step of using NIR measuring to automatically monitor FCC processing conditions.

The process also may include the step of using NIR measuring to maximize FCC monitoring as feedstock parameter changes.

Another embodiment includes the step of using NIR measuring of FCC feed rate, reactor temperature, feed preheat or feed pressure to optimize FCC product monitoring.

Further the step of using NIR measuring of FCC feed parameters to monitor weight percent of each hydrocarbon class may be used. Still further, the step of using NIR measuring to monitor on-line a multiplicity of parameters for FCC processing may be used.

Over 300 FCC feed stocks from multiple refineries were analyzed on a Near Infrared instrument operating between 1100-2500 nm while primary lab results were obtained from the MPC Refining Analytical and Development Laboratory. Collected spectra were imported into FOSS Vision software to perform math functions and multivariate regression analysis. Partial Least Squares regression equations were generated for 20 properties. These properties included core aromatics, distillation points, total and basic nitrogen, sulfur, API gravity, and % Coker Gas Oil. The NIR results can be used in FCC simulation software to predict unit yields and qualities. The NIR provides a 20× savings in labor costs over the conventional lab methods and a significant reduction in analysis time. This time savings would allow for quicker characterization of purchased gas oil.

According to the invention, infrared (preferably NIR) analysis surprisingly has been found capable of predicting the product slate resulting from a particular FCC feed under specified conditions, e.g., cracking severity conditions. Moreover, it has been found that infrared analysis of FCC feed can be made and compared with a model feed and the differences therebetween correlated with catalytic cracking process parameters, e.g., cracking severity, in order to provide economical operation and desired product slates. Variations in the feedstock can affect conversion, product distributions, product properties, operating conditions of the unit, and refinery economics.

### FCC Process

Catalytic cracking is the backbone of many refineries. It converts heavy feeds (600°-1050° F.) such as atmospheric gas oil, vacuum gas oil, coker gas oil, lube extracts, and slop streams, into lighter products such as light gases, olefins, gasoline, distillate and coke, by catalytically cracking large molecules into smaller molecules. Catalytic cracking operates at low pressures (15 to 30 psig), in the absence of externally supplied H₂, in contrast to hydrocracking, in which H₂ is added during the cracking step. Catalytic cracking is inherently safe as it operates with very little oil actually in inventory during the cracking process.

FCC feedstocks include that fraction of crude oil which boils at 650° to 1000° F., such fractions being relatively free of coke precursors and heavy metal contamination. Such feedstock, known as “vacuum gas oil” (VGO) is generally prepared from crude oil by distilling off the fractions boiling below 650° F. at atmospheric pressure and then separating by further vacuum distillation from the heavier fractions a cut boiling between 650° F. and 900° to 1025° F. The fractions boiling above 9000 to 1025° F. are normally employed for a variety of other purposes, such as asphalt, residual fuel oil, #6 fuel oil, or marine Bunker C fuel oil. However, some of these higher boiling cuts can be used as feedstocks in conjunction with FCC processes which utilize carbo-metallic oils by Reduced Crude Conversion (RCC) using a progressive flow type reactor having an elongated reaction chamber.

The FCC process may be controlled by selecting a feedstock of specified characteristics to the unit as well as controlling process parameters.

Varying process conditions can affect the product slate. Operating under more severe cracking conditions by increasing process temperatures can provide a gasoline product of higher octane rating, while increasing conversion can provide more olefins for alkylate production, as well as more gasoline and potential alkylate. Catalytic cracking can also be affected by inhibitors, which can be naturally present in the feed or added separately. Generally, as boiling range of the feed increases, so does the concentration of inhibitors naturally therein. Inhibition effect can be temporary or permanent depending on the type of inhibitor present. Nitrogen inhibitors generally provide temporary effects while heavy metals such as nickel, vanadium, iron, copper, etc., which can quantitatively transfer from the feed to the catalyst provide more permanent inhibition. Metals poisoning results in higher dry gas yields, higher hydrogen factor, higher coke yields as a percent of conversion, and lower gasoline yields. Coke precursors such as asphaltenes tend to break down into coke during cracking which deposits on the catalyst, reducing its activity.

In catalytic cracking, an inventory of particulate catalyst is continuously cycled between a cracking reactor and a catalyst regenerator. In the fluidized catalytic cracking (FCC) process, hydrocarbon feed contacts catalyst in a reactor at 425°-600° C., usually 460°-560° C. The hydrocarbons crack, and deposit carbonaceous hydrocarbons or coke on the catalyst. The cracked products are separated from the coked catalyst. The coked catalyst is stripped of volatiles, usually with steam, and is then regenerated. In the catalyst regenerator, the coke is burned from the catalyst with oxygen-containing gas, usually air. Coke burns off, restoring catalyst activity and heating the catalyst to, e.g., 500°-900° C., usually 600°-750° C. Flue gas formed by burning coke in the regenerator is discharged into the atmosphere.

Many FCC units now use zeolite-containing catalyst having high activity and selectivity. These catalysts are generally believed to work best when the coke on catalyst after regeneration is relatively low, say, less than 0.1 wt %, and preferably less than 0.05 wt %.

To regenerate FCC catalysts to these low residual carbon levels, and to burn CO completely to CO₂ within the regenerator (to conserve heat and minimize air pollution) many FCC operators have turned to high efficiency regenerators and to CO combustion promoters. Many FCC units operate in complete CO combustion mode, i.e., the mole ratio of CO2/CO is at least 10. Refiners burn CO within the regenerator to conserve heat and minimize air pollution. The preferred way to burn CO in the regenerator is to add platinum catalyst.

Various methods of practicing the present invention can be carried out. In one embodiment, a product slate can be determined based upon or an IR analysis of cracking product. This data can then be used for unit monitoring.

Alternatively, the feed may be characterized based upon an IR analysis of the feed. This data can then be used for unit monitoring.

Feed variables which may be used in the present invention are selected from the group consisting of wt. % or vol. % monoaromatics, diaromatics, triaromatics, benzothiophenes and dibenzothiophenes, paraffins/naphthenes, aromatics, nitrogen content, and the like.

Product variables which may be used in the present invention are selected from the group consisting of C4 free gasoline (vol) total C4's (vol) dry gas (wt), coke (wt), gasoline octane, LFO, HFO, H2S, sulfur in LFO, aniline point of LFO and the like.

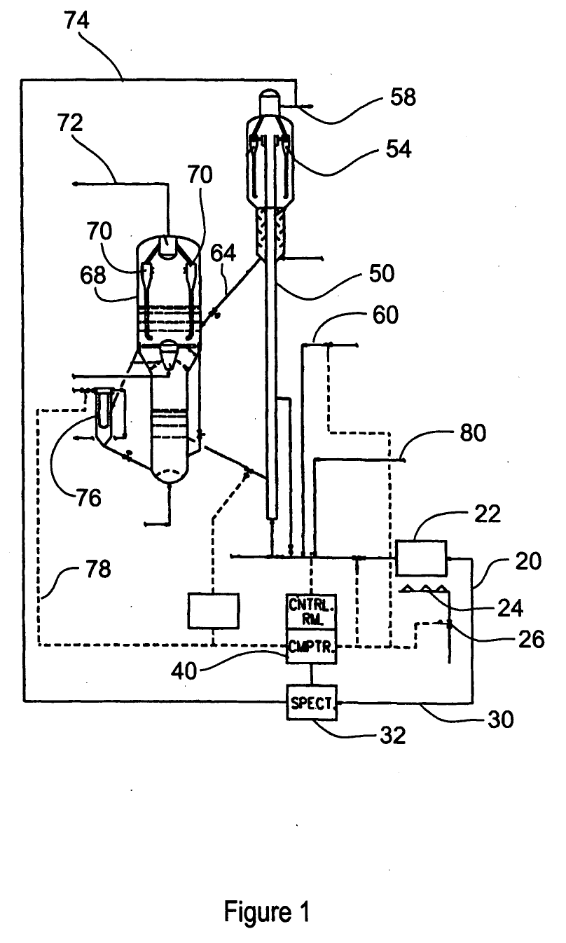

FIG. 1 is a schematic diagram of an FCC unit comprising a reactor and a regenerator showing the monitoring system of the present invention in place for operating that FCC unit.

FIG. 1 shows feed 20 is heated by fired heater 22 which is heated by gas burner 24, fuel to which is controlled by automatic valve 26. Just before the fuel enters the fired heater 22, a sample 30 is withdrawn and conducted by tubing into NIR unit 32. In an alternate embodiment (not shown), a fiber optic probe inserted directly into the feed line before fired heater 22 can obviate the need for withdrawing sample.

NIR unit 32 can be located in a laboratory, at-line or on-line and can include a sample conditioning means for controlling the temperature, and for extracting bubbles and dirt from the sample. The NIR unit also comprises a spectrometer means which may be a spectrometer of the NIR, Fourier Transform Near Infrared (FTNIR), Fourier Transform Infrared (FTIR), or Infrared (IR) type, ruggedized for process service and operated in a temperature-controlled, explosion-proof cabinet. A photometer with preset optical filters moving successively into position, can be used as a special type of spectrometer.

The spectrometer 32 outputs a signal to computer 40 which preferably takes a derivative of the signal from the spectrometer, and subjects it to a defined model to generate the properties of interest. The model is optionally derived from signals obtained from NIR measurement of cracking products.

In operation, the FCCU operates conventionally with feed being fired in heater 22 entering riser 50, together with catalyst descending through the catalyst return line 52 and entering riser 50. The vaporized products ascend riser 50 and are recovered in the reactor by cyclone 54 with product vapors 58 exiting to the main column for fractionation and recovery of various products. Naphtha product can be recycled through line 79. Spent catalyst descends from the reactor through lines 64 into the regenerator 68 and contacts air to burn off carbon and produce flue gas which exits through flue cyclone 80 and flue gas line 84.

Optionally or alternatively, a second sample taken from the reactor product vapors 58 can be input through line 59 to spectrometer 32, permitting the spectrometer to analyze the products so that computer 40 can compare the group type analysis of the products against the optimum products slate desired for maximum economy.

### EXAMPLE 1

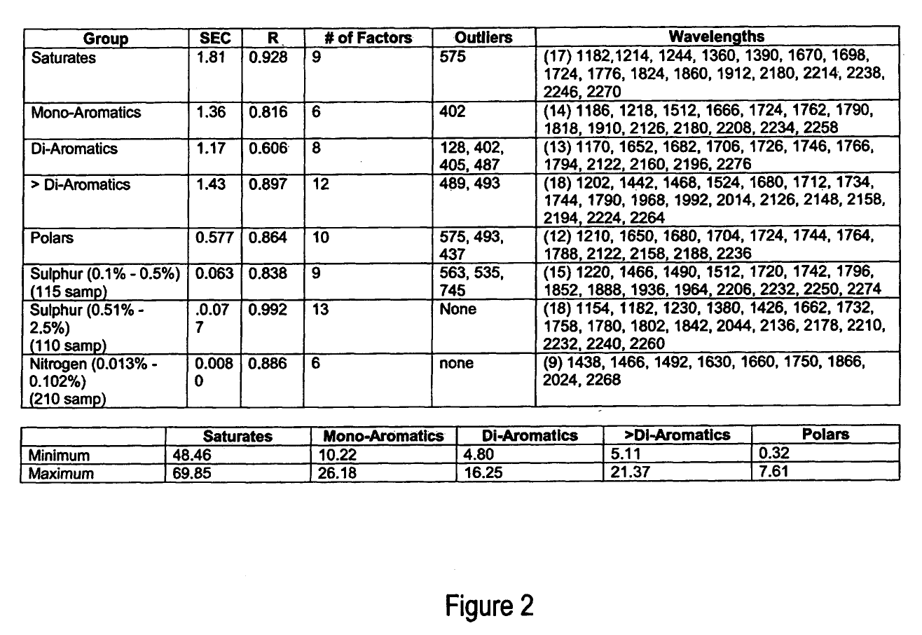

FIG. 2 is a Table which shows samples, including hydrotreater charges and products and FCC feeds used to control weight percents of each hydrocarbon class.

Two hundred fifty samples, including hydrotreater charges and products and FCC feeds were used to create a PLS model for predicting weight percents of each hydrocarbon class. The samples were analyzed using the online NIR. Wavelengths were chosen for each group and a summary appears in FIG. 2.

### EXAMPLE 2

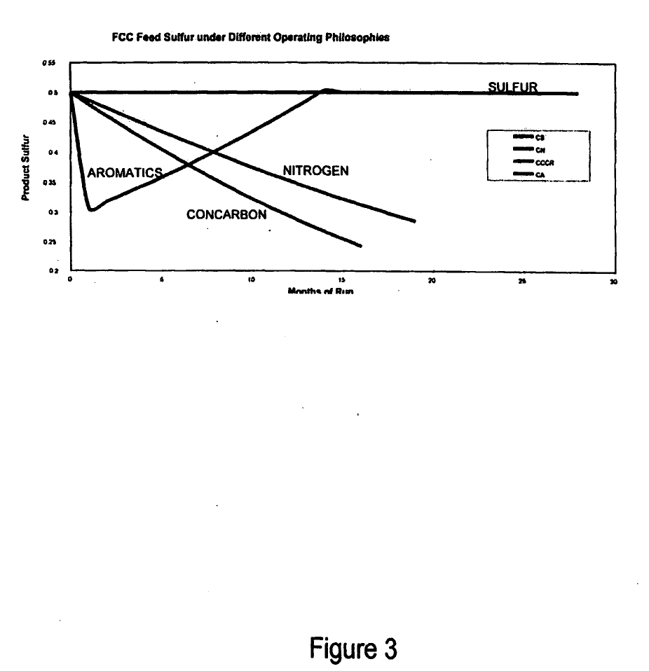

FIG. 3 is a plot that illustrates HDS vs. AS mode differences. The plot shows FCC feed sulfur under different operating philosophies. The feed sulfur is held constant and aromatics, nitrogen or concarbon parameters are varied.

### EXAMPLE 3

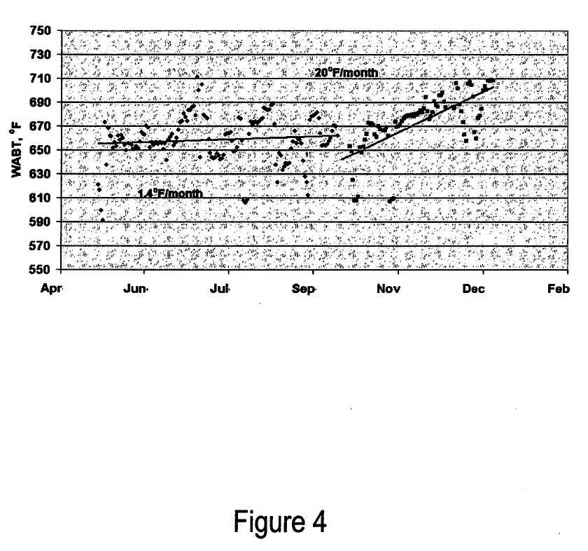

FIG. 4 is a graph of a catalyst cycle life curve. A critical aspect of managing the CFH is catalyst cycle life. Coke and metals are deposited on the catalyst during the course of the run cycle. This deactivation requires an increase in temperature. End of Run is typically determined when the process is at its maximum inlet temperature capability. At this point the catalyst will need to be changed out with fresh. Monitoring the CFH feed properties will ensure the unit is managed to achieve the desired cycle length and avoid an upset condition where poor feed quality is sent to the unit. This ability to monitor feed provides for greater flexibility and minimizes risk for increased deactivation and catalyst damage. FIG. 4 is a typical catalyst cycle life curve showing the impact of a feed upset. In this case the upset was caused by a leaking heat exchanger. Application of the NIR for on-line feed monitoring would allow better unit monitoring to minimize the risk of this type of upset.

### EXAMPLE 4

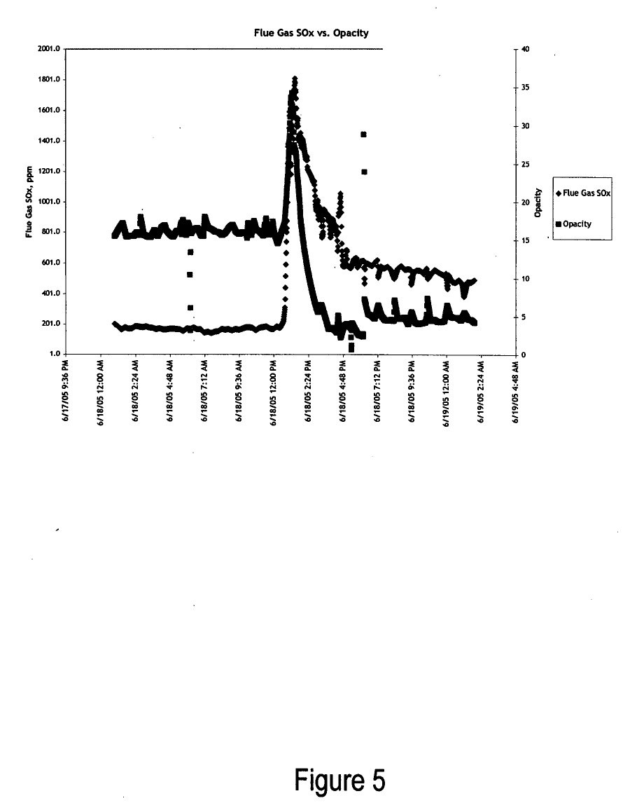

FIG. 5 is a graph of an FCC feed upset showing high SO_{X}. All FCC units have environmental emission limits. These are typically SO_{X}, NO_{X}, CO and particulate matter. Advanced monitoring of FCC feed properties for S and N will enable the refiner to adjust process conditions to ensure a feed change or upset will not cause an environmental exceedence. Operating actions may include decreasing federate, changing feed line-ups, diverting certain feed streams, and adjusting catalyst additive use for SO_{X} and NO_{X}. FIG. 5 is an example of an FCC feed upset resulting in high SO_{X} and opacity. Use of NIR on the FCC feed stream would provide advanced notice of the pending problem and enable the operator to take action to mitigate.

### EXAMPLE 5

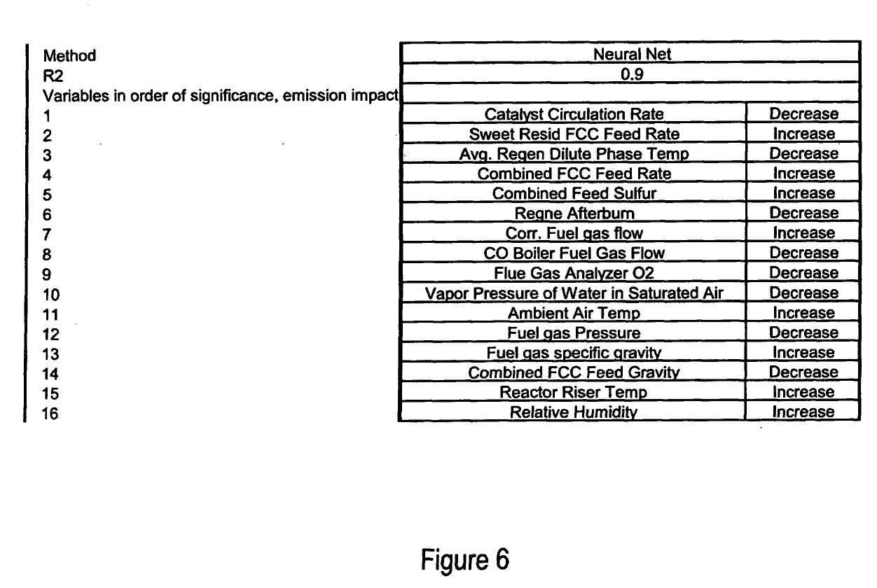

FIG. 6 is a table of a neural network for on-line control of SO_{X} emissions. The NIR analyzer on the FCC feed can also be used to model FCC emissions. Several refiners have developed either statistical or neural network models to predict FCC emissions either with or without catalyst additives. Use of the NIR to measure feed characterization would be an important new parameter to improve model accuracy. Current models are developed based upon daily feed samples that often result in poor correlations due to variability. FIG. 6 is a summary of a neural network model variables use to predict SO_{X} emissions on the FCC unit at a refinery. NIR would allow for improved monitoring of the Feed properties and improve the model's capacity.

### EXAMPLE 6

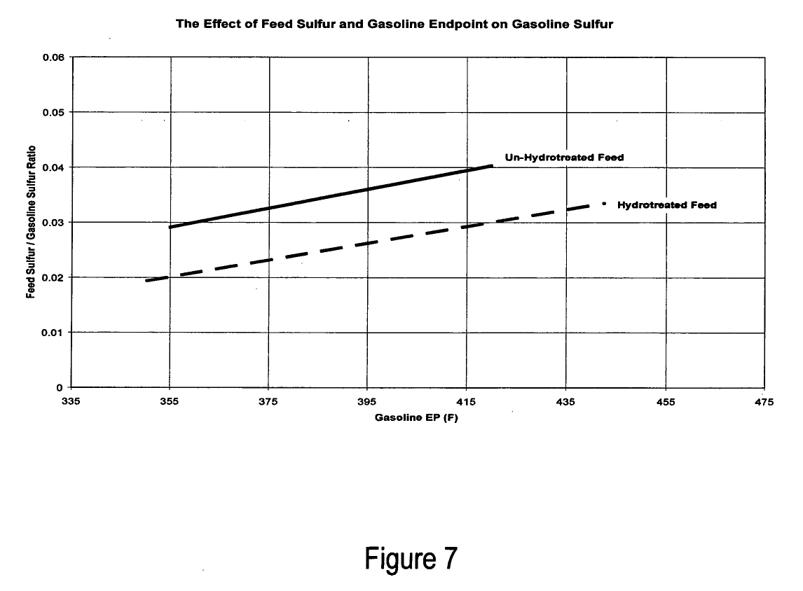

FIG. 7 is a graph of the use of NIR on FCC hydrotreating. Refiners have had to choose between pre-treat and post-treat options to meet gasoline sulfur requirements. Units that rely on controlling FCC feed sulfur via pre-treating with a CFH will see variation in the feed sulfur to gasoline sulfur ratio with different crude types, CFH operating conditions and degree of hydroprocessing. In order to ensure gasoline product quality, it is important to ensure gasoline sulfur content is controlled. Use of NIR on the FCC feed would allow the unit to adjust processing conditions to maintain product quality and avoid an off-spec product. On-line monitoring of FCC feed quality will allow the operator to adjust the CFH severity, change FCC federate, divert certain feed streams and adjust product fractionation to maintain product quality. FIG. 7 is a typical relationship between feed sulfur, gasoline sulfur and gasoline endpoint. The NIR capability would allow the process to stay at the control point of this curve.

### EXAMPLE 7

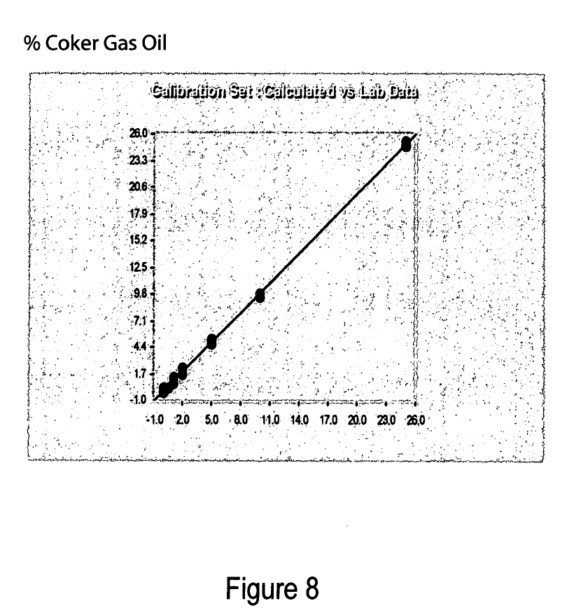

FIG. 8 is a graph showing NIR predicted results versus Lab results for sulfur, API gravity, and % Coker Gas Oil.

The results show that it has been found possible to be able to predict feed properties from a detailed description of the catalytic cracking feed composition by hydrocarbon group types (HGT), which can affect catalytic cracking operations and products. The properties can be related to the weighting of certain components in the composition of the cracking feed, e.g., monoaromatics, diaromatics, triaromatics, benzothiophenes and dibenzothiophenes. HGT is determined by the techniques illustrated in the Table in FIG. 2 and from changes in specific near infrared absorption bands. Correlating the content of these components in the feed to FCC product properties can be accomplished using the process of this invention for unit monitoring.

### EXAMPLE 8

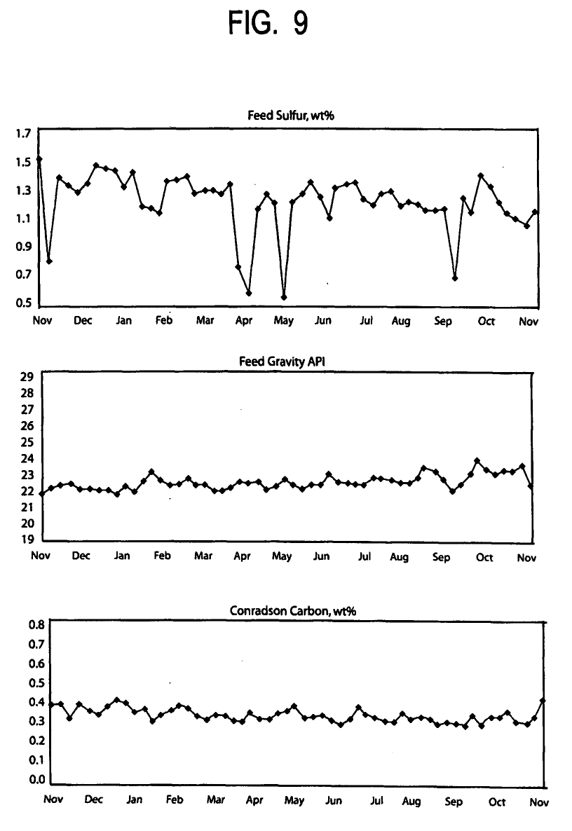

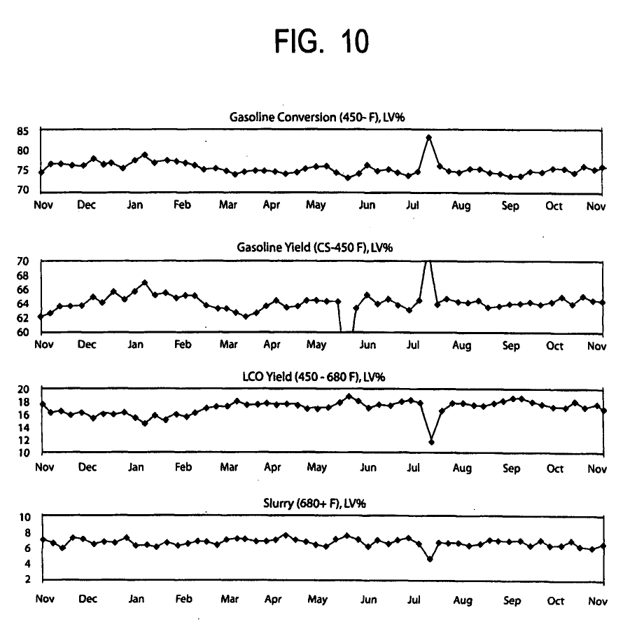

FIGS. 9 and 10 show graphs of typical unit monitoring plots used to track feedstock quality and product yields. Use of the NIR will allow this be done on-line or in the lab and provide better resolution. FIG. 9 shows weight percent data for Feed Sulfur, Feed Gravity and Contradson Carbon. FIG. 10 shows weight percent data for Gasoline Conversion, Gasoline Yield, LCO yield, and slurry LV %.

### Modifications

Specific compositions, methods, or embodiments discussed are intended to be only illustrative of the invention disclosed by this specification. Variation on these compositions, methods, or embodiments are readily apparent to a person of skill in the art based upon the teachings of this specification and are therefore intended to be included as part of the inventions disclosed herein.

The above detailed description of the present invention is given for explanatory purposes. It will be apparent to those skilled in the art that numerous changes and modifications can be made without departing from the scope of the invention. Accordingly, the whole of the foregoing description is to be construed in an illustrative and not a limitative sense, the scope of the invention being defined solely by the appended claims.

We claim:

1. A process for analyzing catalytic cracking hydrocarbon feeds intermediates and products exhibiting absorption in the near infrared (NIR) region comprising:
a) measuring absorbances of said feed, intermediates, or products using a spectrometer measuring absorbances at wavelengths within the range of about 780-4000 nm, and outputting an emitted signal indicative of said absorbance;
b) subjecting the NIR spectrometer signal to a mathematical treatment (e.g., derivative, smooth, baseline correction) of the emitted signal.
c) processing the emitted signal or the mathematical treatment using a defined model to determine the chemical or physical properties of feeds, intermediates or products and outputting a processed signal; and
d) monitoring on-line in response to the processed signal, at least one parameter of the catalytic cracking feed, intermediate or product.

2. The process of claim 1 including the step of using NIR measuring to provide real time optimization of (RTO) FCC monitoring.

3. The process of claim 1 including the step of using NIR measuring to automatically monitor FCC processing conditions.

4. The process of claim 1 including the step of using NIR measuring to maximize FCC monitoring as feedstock parameter changes.

5. The process of claim 1 including the step of using NIR measuring of FCC feed rate, reactor temperature, feed preheat or feed pressure to optimize FCC product monitoring.

6. The process of claim 1 including the step of using NIR measuring of FCC feed parameters to monitor weight percent of each hydrocarbon class.

7. The process of claim 1 including the step of using NIR measuring to monitor on-line a multiplicity of parameters for FCC processing.

8. The process of claim 1 wherein said absorbances are measured at wavelengths within the range of about 780-2500 nm.

9. The process of claim 1 wherein said absorbances are measured at wavelengths within the range of 1100-2200 nm.

10. The process of claim 1 wherein said absorbance is measured in at least one wavelength and includes the steps of:
a) periodically or continuously outputting a periodic or continuous signal indicative of the intensity of said absorbance in said wavelength, or wavelengths in said two or more bands or a combination of mathematical functions thereof, and
b) mathematically converting said signal to an output signal indicative of the mathematical function.

11. The process of claim 1 wherein said feed, intermediate, or product are measured for content of at least one of monoaromatics, diaromatics, triaromatics, tetraaromatics, polar aromatics, total aromatics benzothiophenes, dibenzothiophenes, distillation points, basic nitrogen, total nitrogen, API gravity, total sulfur, MCRT and % coker gasoil.

12. The process of claim 1 wherein the catalytic cracking produces products having lower average molecular weight than the feed, by contacting the feed with catalyst in a contacting zone and recovering and separating the products exiting from the cracking zone.

13. The process of claim 1 wherein the parameter of catalytic cracking of step (c) is selected from the group consisting of temperature, throughput, pressure, hydrogen feed rate, catalyst, and oil ratio.

14. The process of claim 1 including the steps of:
obtaining a first data set of NIR spectroscopic data samples by subjecting the feed, intermediates, or products to NIR spectroscopy;
generating a second data set of NIR spectroscopic data samples by processing the first data set using a second technique; and
identifying a component of the feed by performing a NIR analysis on the second data set.

15. The process of claim 1 including the step of:
mathematically converting the signal to an output signal indicative of the parameter.

16. The process of claim 15 including the steps of:
periodically or continuously outputting a periodic or continuous signal indicative of the intensity of the NIR absorbance in the wavelength, or wavelengths in the two or more bands or a combination of mathematical functions thereof, and
mathematically converting said signal to an output signal indicative of the mathematical function.

17. The process of claim 1 including the step of using the NIR results in FCC simulation software to predict unit yields and qualities.

18. The process of claim 14 which allows direct monitoring of the feedstock properties and effluent yields in real time to ensure product quality and processing targets are achieved.
